(19) United States
(12) Patent Application Publication
(10) Pub. No.: US 2021/0045816 A1
(43) Pub. Date: Feb. 18, 2021

(54) SPINAL ORIENTATION SYSTEM

(71) Applicant: Integrity Implants Inc., Palm Beach Gardens, FL (US)

(72) Inventor: Wyatt Drake Geist, Davie, FL (US)

(21) Appl. No.: 16/991,402

(22) Filed: Aug. 12, 2020

Related U.S. Application Data
(60) Provisional application No. 62/885,412, filed on Aug. 12, 2019.
(60) Provisional application No. 62/889,758, filed on Aug. 21, 2019.

(51) Int. Cl. A61B 34/30; A61B 17/70; A61B 90/00; A61B 34/20; A61B 6/00

(52) U.S. Cl. CPC A61B 34/30; A61B 17/7076; A61B 90/39; A61B 34/20; A61B 2034/2055; A61B 2090/3916; A61B 2090/3937; A61B 2090/3983; A61B 6/4441

(57) ABSTRACT

The invention involves a system and method for confirming the orientation of the spine around and along the longitudinal axis of the spine to provide additional accuracy with pedicle screw placement when the pedicle screw placement is by hand or with a robot. The system utilizes a CT scan of the patient which is overlain with a real time fluoroscopic image to confirm the proper orientation and positioning. Optical or electromagnetic markers can then be utilized to monitor for movement of the spine during the procedure.

### RELATED APPLICATIONS

In accordance with 37 C.F.R. 1.76, a claim of priority is included in an Application Data Sheet filed concurrently herewith. Accordingly, the present invention claims priority to U.S. Provisional Application No. 62/885,412, filed Aug. 12, 2019, entitled “Spinal Orientation System”, as well as U.S. Provisional Application No. 62/889,758, filed Aug. 21, 2019, entitled “Spinal Orientation System”; the contents of which are incorporated herein by reference.

### FIELD OF INVENTION

The present invention generally relates to medical imaging and, more particularly, to a system for determining an angle of rotation of the spine about its longitudinal axis with respect to a perpendicular axis.

### BACKGROUND INFORMATION

Fluoroscopy machines are often used in hospital emergency rooms and trauma centers. These machines have an arm which supports an x-ray source spaced apart from an x-ray detector. The arm, generally a C-shaped arm, is utilized to locate the x-ray source with respect to the x-ray detection; and can be manipulated to place the x-ray source on one side of a patient and the x-ray detector on the other side of the patient. A series of joints permit the arm to be manually moved to a pose which will provide a desired x-ray image. A monitor displays the x-ray image in real time. C-arm fluoroscopy machines may, for example, be used to image the locations at which pins or screws will be inserted to hold bones in position.

One issue with respect to C-arm fluoroscopy machines is that they lack a definite datum angle with respect to the patient's spine. In other words, the patient, and thus their spine, could be rotated a few degrees to either side when the surgeon assumes the spine is oriented in the desired alignment with the surgical table. This rotation may cause the pedicle screws to be inserted at an incorrect angle with respect to the pedicle, causing a medial or lateral breach.

An additional drawback to the prior art relates to the use of robots to introduce apertures for pedicle screws or for insertion of pedicle screws. Robots assume the vertebrae are oriented with the transverse process arranged horizontally and the spinous process oriented vertically. In this case, rotation of the spine along its longitudinal axis and with respect to a theoretical vertical plane bisecting the vertebrae may reduce the tolerance usable by the robot to prevent breach of the vertebrae with a pedicle screw.

Thus, the present system provides a method of checking the rotational relationship of the spine about its longitudinal axis, which overcomes the disadvantages of prior art surgical methods. The present spinal orientation system not only provides for accuracy, it also permits cross checking of the orientation with visual and/or electromagnetic sensors, along with visual indicators comparing CT scans with fluoroscopy scans.

### SUMMARY OF THE INVENTION

Briefly, the invention involves a system and method for confirming the orientation of the spine around and along the longitudinal axis of the spine to provide accuracy with pedicle screw placement when the pedicle screw placement is by hand or with a robot. The system utilizes a CT scan of the patient, which is overlaid with a real time fluoroscopic image to confirm the proper orientation and position. Optical or electromagnetic markers can then be utilized to monitor for movement of the spine during the surgical procedure.

Accordingly, it is an objective of the present invention to provide a system for confirming the orientation of the spine about and along a longitudinal axis for spinal procedures.

It is a further objective of the present invention to provide a system for confirming the orientation of the spine that utilizes a CT scan and real time fluoroscopy.

Other objectives and advantages of this invention will become apparent from the following description taken in conjunction with the accompanying drawings wherein are set forth, by way of illustration and example, certain embodiments of this invention. The drawings constitute a part of this specification, include exemplary embodiments of the present invention, and illustrate various objects and features thereof.

### BRIEF DESCRIPTION OF THE FIGURES

FIG. 1 is a schematic view illustrating one embodiment of the spinal orientation system of the present invention;

FIG. 2 is a partial rear view of a vertebra illustrating a portion of the lumbar spine;

FIG. 3 is a partial rear view of a vertebra illustrating a portion of the thoracic spine;

FIG. 4 is an end view illustrating a thoracic vertebra at the T3 level;

FIG. 5 is an end view illustrating a thoracic vertebra at the T1 level;

FIG. 6 is a side view of a vertebra illustrating cranio caudal angulation of pedicle screw placement;

FIG. 7 is a side view of a vertebra illustrating a pedicle screw placed in the vertebra;

FIG. 8 is an end view of a pedicle illustrating a lateral breach of the pedicle screw;

FIG. 9 is an end view of a pedicle illustrating a medial breach of the pedicle screw;

FIG. 10 is an end view of a pedicle illustrating an accurate placement of the pedicle screw;

FIG. 11 illustrates a fluoroscopy image taken to locate the sagittal plane of the vertebra; and

FIG. 12 illustrates a CT scan image of the same area of the fluoroscopy image of FIG. 11.

### DETAILED DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated.

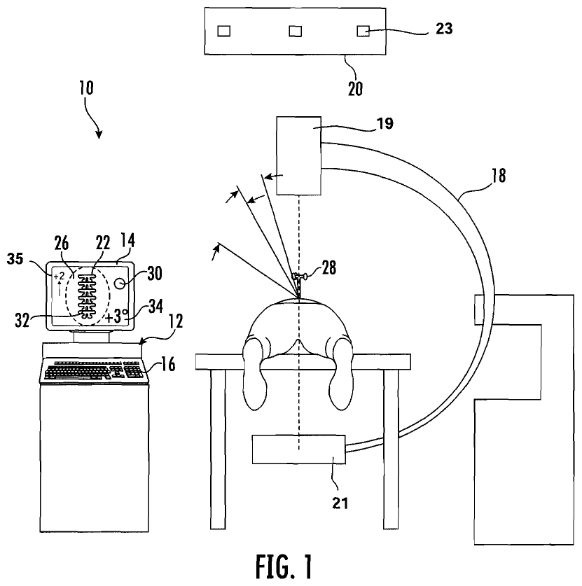

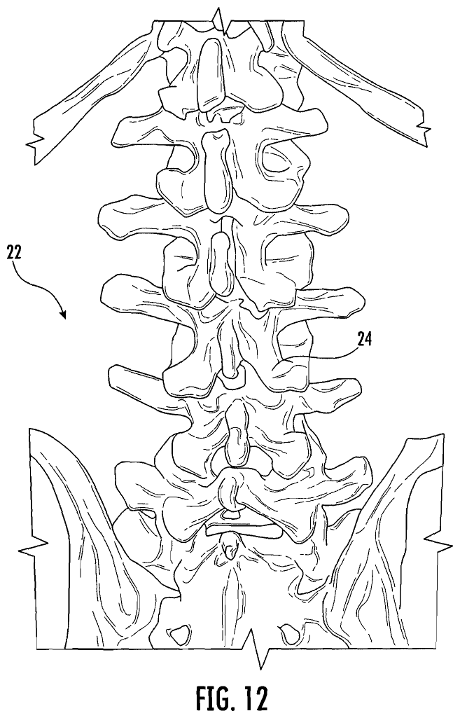

Referring generally to FIGS. 1-11, a spinal orientation system 10 for determining the rotational orientation of the spine about and along the longitudinal axis is illustrated. The system generally includes a computer 12, a monitor 14, a keyboard 16, a C-arm 18 and an optical or electromagnetic monitoring system 20. The computer 12 includes a processor (not shown) and sufficient memory to contain and display a computed tomography (CT) scan image 22, magnetic resonance (MRI) image or the like of the spine 24 (FIG. 12). A C-arm 18 is also connected to the computer 12 for input of a fluoroscopy image 26; the C-arm 18 including an x-ray source 19 positioned at a first end of said C-arm 18 and an x-ray detector positioned at a second end of said C-arm. The computer memory having a stored file including a CT or MRI image stored thereon for recall onto the monitor 14 for viewing. The spinal orientation system 10 is preferably constructed and arranged to overlay the CT scan image 22 or MRI scan image over the fluoroscopy image 26. However, in an alternative embodiment, the fluoroscopy image 26 can be overlaid onto the CT scan image or MRI scan image without departing from the scope of the invention. In this manner, the CT image 22 or MRI image can be oriented so that the sagittal plane or any other established plane is horizontal or vertical for comparison to the fluoroscopy image 26. If the images match, an optical sensor 28 can be attached to a portion of the spine 24 to allow the optical monitoring system 20 to monitor the spine for movement. Such optical monitoring systems 20 are well known in the medical art. In such optical monitoring systems, one or more optical sensors 28 are attached to a bone such as a vertebra within the viewing frame of one or more optical monitoring cameras 23 and alert the surgeon if movement is detected. Once movement is detected, or in the event that the overlaid CT image 22 or MRI images and the fluoroscopy images 26 do not match, a colored indicator 30 or reference line 32 will be illustrated on the monitor 14 to indicate to the surgeon that the spine is rotated or translated with respect to the vertical axis created by the sagittal or otherwise established plane. In this scenario, the CT or MRI image, and thus the sagittal or otherwise established plane, can be rotated about the longitudinal axis or translated along the spine until the images do match. The number of degrees that the CT image or MRI image was rotated with respect to the fluoroscopy image(s) 26 can then be indicated to the physician as a rotation angle 34. Once the rotation angle 34 is determined, the surgeon can use the rotation angle 34 to place the screws with a robot (not shown) or by hand. Likewise, the distance that the image is translated along the longitudinal axis can be indicated as a translation distance 35 and utilized for location of the pedicle screw entry point.

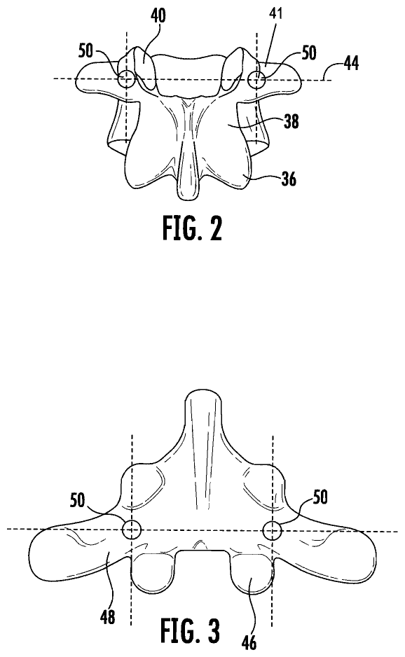

Referring to FIGS. 2 and 3, the entry point of a pedicle screw into a lumbar vertebra is illustrated. The entry point is generally defined as the confluence of any of four lines, e.g. the pars interarticularis 38, the mammillary process 41, the lateral border of the superior articular facet 40, and the mid transverse process 44.

Referring to FIG. 3, the entry point of a pedicle screw into the lower thoracic segments is generally determined by the mid portion of the facet joint 46 and the superior edge of the transverse process 48. The specific entry point would preferably be just lateral and caudal to this intersection.

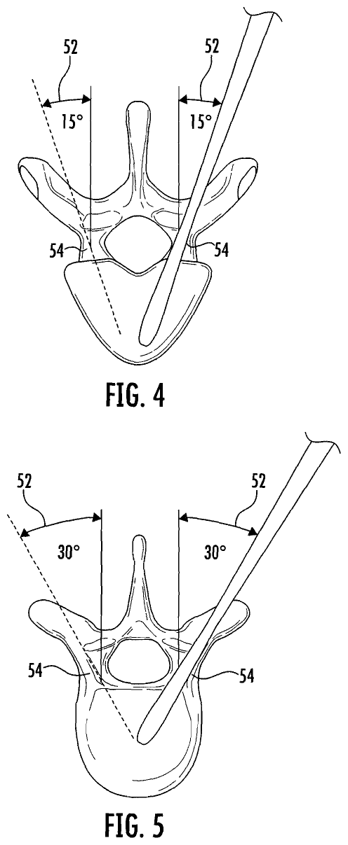

Referring to FIGS. 4 and 5, the mediolateral inclination is illustrated. The mediolateral inclination will depend upon the rotation of the vertebra around the longitudinal axis of the spine. The main goal is to prevent medial penetration of the spinal canal superficially, and lateral or anterior penetration of the vertebral body cortex at the depth of insertion. Ideally, two screws should converge but stay entirely within the cortex of the pedicles and body. As illustrated, the transverse angle 52 of the pedicle 54 ranges in angulation from about 30 degrees at the T1 level to about 15 degrees at the T3 level, and from T4 downward, the transverse angle is almost sagittal.

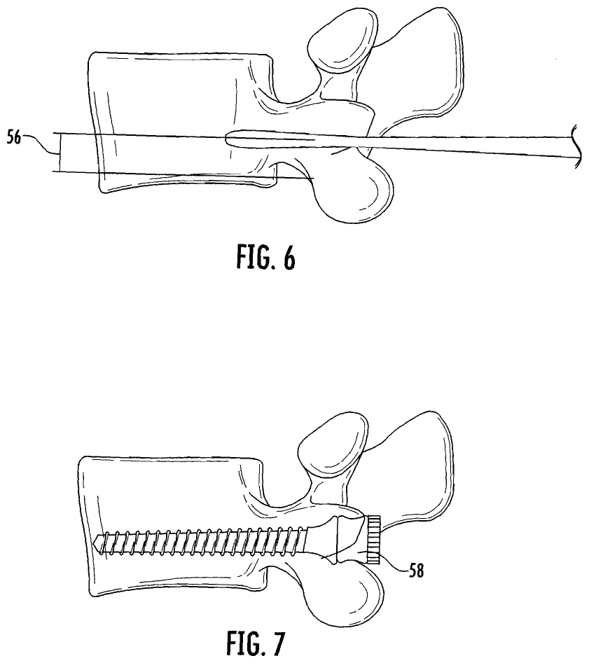

Referring to FIGS. 6 and 7, the cranial-caudal angulation 56 is illustrated. The appropriate trajectory is aiming for the contralateral transverse process. FIG. 7 illustrates proper placement of a pedicle screw 58.

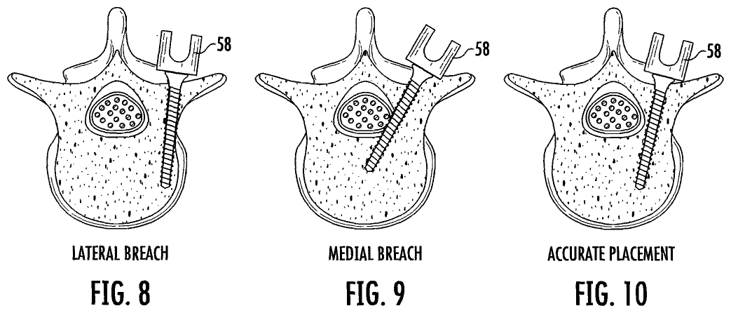

FIGS. 8-10 illustrate various placements of pedicle screws 58. FIG. 8 illustrates a lateral breach of the pedicle screw 58. FIG. 9 illustrates a medial breach of the pedicle screw 58. FIG. 10 illustrates a properly placed pedicle screw 58.

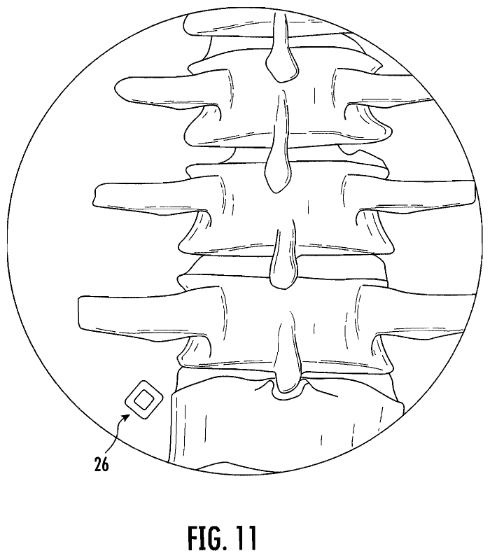

Referring to FIGS. 11 and 12, a fluoroscopy image 26 and a CT scan image 22 or MRI scan image are illustrated. The images, while not specific to the invention, are representative of the type of images taken for spinal surgery.

It is to be understood that while a certain form of the invention is illustrated, it is not to be limited to the specific form or arrangement herein described and shown. It will be apparent to those skilled in the art that various changes may be made without departing from the scope of the invention, and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention, which are obvious to those skilled in the art, are intended to be within the scope of the following claims.

What is claimed is:

1. A spinal orientation system for determining the rotational orientation of the spine about and along the longitudinal axis comprising:
a computer, said computer including a processor and sufficient memory to store and display at least one image of a spine, a monitor in electric communication with said computer for display of said at least one image of a spine, a keyboard in electric communication with said computer for input of commands to said computer for display of said at least one image of a spine, a C-arm in electric communication with said computer, said C-arm including an x-ray source positioned at a first end of said C-arm and an x-ray detector positioned at a second end of said C-arm to take a fluoroscopic image of a spine for input of said fluoroscopic image to said computer and display of said fluoroscopic image on said monitor, said computer memory having a stored file including a computer tomography image stored thereon for recall onto the monitor for viewing via said keyboard, said system constructed and arranged so that said computer tomography image can be overlaid onto said fluoroscopic image on said monitor whereby both images are simultaneously viewable, said computer tomography image moveably positionable to align with said fluoroscopic image.

2. The spinal orientation system of claim 1 wherein said computer tomographic image is aligned with a predetermined plane of said fluoroscopic image.

3. The spinal orientation system of claim 2 wherein said computer tomographic image is aligned with a sagittal plane of said fluoroscopic image.

4. The spinal orientation system of claim 1 wherein said computer tomographic image is a magnetic resonance image.

5. The spinal orientation system of claim 4 wherein said fluoroscopic image is overlaid onto said computer tomographic image.

6. The spinal orientation system of claim 1 wherein said spinal orientation system further includes an optical monitoring system for monitoring for spinal movement, said optical monitoring system in electrical communication with said computer for providing a visible alert to detected movement on said monitor to indicate to the surgeon that a portion of a spine has rotated or translated with respect to said predetermined or said sagittal plane.

7. The spinal orientation system of claim 6 wherein said visible alert is a colored indicator.

8. The spinal orientation system of claim 6 wherein said visible alert is a reference line indicating the area of movement.

9. The spinal orientation system of claim 6 wherein said optical monitoring system includes at least one optical sensor securable to a bone.

10. The spinal orientation system of claim 9 wherein said optical monitoring system includes one or more optical monitoring cameras constructed and arranged to monitor movement of said at least one optical sensor.

11. The spinal orientation system of claim 1 wherein said alignment of said computer tomographic image provides a rotation angle of said fluoroscopic image with respect to said computer tomographic image.

12. The spinal orientation system of claim 11 wherein said rotation angle adds to or subtracts from about 30 degrees at the T1 level and from about 15 degrees at the T3 level, and from a sagittal angle from T4 downward.

13. The spinal orientation system of claim 1 wherein said alignment of said computer tomographic image provides a translation distance of said fluoroscopic image with respect to said computer tomographic image for use as a pedicle screw entry point.

14. The spinal orientation system of claim 13 wherein said entry point is defined as the confluence of any of four reference lines, including the pars interarticularis, the mammillary process, the lateral border of the superior articular facet, and the mid transverse process.

15. The spinal orientation system of claim 13 wherein said entry point into a lower thoracic segment is generally determined by a mid portion of a facet joint and a superior edge of a transverse process.
